(19) United States
(12) Patent Application Publication
(10) Pub. No.: US 2009/0005782 A1
(43) Pub. Date: Jan. 1, 2009

(54) Fracture Fixation System and Method

(76) Inventors: Paul E. Chirico, Campbell, CA (US); Benny M. Chan, Fremont, CA (US); R. Sean Pakbaz, San Diego, CA (US); Joseph A. Horton, Hoover, AL (US); Alison A. Souza, Santa Clara, CA (US); Brian E. Martini, Aptos, CA (US)

Correspondence Address: SHAY GLENN LLP, 2755 CAMPUS DRIVE, SUITE 210, SAN MATEO, CA, 94403, US

(21) Appl. No.: 12/041,607

(22) Filed: Mar. 3, 2008

Related U.S. Application Data
(60) Provisional application No. 60/904,578, filed on Mar. 2, 2007.

(51) Int. Cl. A61B 17/56; A61B 17/04

(52) U.S. Cl. 606/63; 606/300

(57) ABSTRACT

The invention relates to a system for stabilizing a bone fracture and methods for applying the system. The system includes a device with two anchorable members with an intervening connector and a passageway through the device. The anchorable members have a constrained non-anchoring configuration and a released anchoring configuration. The anchoring configuration includes a radially-expanded structure such as a plurality of struts. After implantation across a fracture site, the anchorable members are released from their linearly constrained configuration, and structural features radially self-expand, anchoring the device across the fracture. A flowable bone-filling material may be conveyed into the passageway of the device after implantation. The composition fills the space within the expanded structures of the anchorable members and flows into space surround the device, stabilizing it further in the implantation site.

### CROSS REFERENCE TO RELATED APPLICATIONS

This application claims priority to U.S. Provisional Patent Application No. 60/904,578 of Chirico et al., entitled “Fracture Fixation System and Method”, filed on Mar. 2, 2007.

### FIELD OF THE INVENTION

The invention relates to a system and methods of using the system to securely fix aligned bone fracture segments in place to promote optimal healing of the fracture.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

The goal of bone fracture fixation is to stabilize bone regions around the fracture in an optimal alignment, and by such stabilized and supported alignment, allow fast healing of the fracture, and a return to mobility and function of the fracture and surrounding region as a whole. Fracture fixation methods are generally categorized as external or internal. Internal fixation methods are more interventional and surgical in nature than external fixation methods, and they may also be complemented by the support of external methods. External fixation typically includes a closed reduction to restore or maintain alignment of fractured regions, which is then stabilized by splints, casts, and slings. External traction can also be applied to the fracture, taking advantage of leverage that can be applied to these external structures. Internal fixation methods include the interventional use of various hardware elements such as wires, pins and screws, plates, intramedullary nails or rods, staples, and clamps. Internal fixation devices and approaches have also created an avenue for introducing bioactive agents into the fracture site, such osteoinductive agents or anti-infective agents, that can encourage bone healing and combat infections. Bone fractures are by their nature highly individual and complex. It would therefore be beneficial to provide new devices and methods, particularly those that can readily be tailored to fracture specifics and create minimal collateral disturbance.

### SUMMARY OF THE INVENTION

The invention provided herein relates to a system for stabilizing a bone fracture, and methods for applying that system. The system includes a first anchorable member and a second anchorable member, each member having a central passageway, each member having a constrained non-anchoring configuration and a released anchoring configuration. The system further includes a connector having a central passageway, the connector configured to be attached to the proximal end of the first anchorable member and the distal end of the second anchorable member, such that the central passageways of the anchorable members and the connector form a continuous passageway. The system may further include delivery devices, devices that rotate or otherwise manipulate the system in situ, and devices for injection of bone-filling compositions. The first anchorable member, the second anchorable member and the connector may be collectively referred to as a fracture-stabilizing device.

Embodiments of the system may be configured in various ways with regard to the extent to which the anchorable members and the connector are separate or conjoined. In some embodiments, the anchorable members and the connector are formed as an integral device. In some embodiments, the first and second anchorable members and the connector are all separate elements. In other embodiments, the first anchorable member and the connector are conjoined, and the second anchorable member is separate. In other embodiments the first anchorable member is separate and the connector and the second anchorable member are conjoined. In the embodiments where the anchorable members and the connector are not fully integrated, they may be assembled prior to delivery to a fracture site, or they may be assembled during the delivery and anchoring of the device to the target fracture site.

In typical embodiments, the constrained (e.g., non-anchoring) configuration of an anchorable member is substantially linear in form, and the released (e.g., anchoring) configuration includes a radially expanded structure. In some variations, the non-anchoring configuration of the member includes three or more flat surfaces in cross section; some of these embodiments may have a rectangular cross section. In other embodiments, the member has a rounded configuration in the unexpanded state. In some embodiments, the released configuration with a radially expanded structure includes expandable struts. In various embodiments of the struts, they may present a flat or a rounded surface as a leading edge. More preferably, the struts of the self-expanding members may include a cutting edge that is sharp and sufficiently strong to cut into bone. This leading edge may be a knife-edge, a serrated edge, or the like. In some embodiments, the expandable struts form a symmetrical bow when freely expanded; in other embodiments they may form an asymmetrical bow. In strut embodiments that form an asymmetrical bow, the asymmetry may include a bow that has its greatest radial diameter distributed either distally or proximally.

The passageways of the first anchorable member, the connector, and the second anchorable member may be adapted to convey a flowable material such as a bone filling composition or cement, which may include biological materials, synthetic materials, inorganic materials, or bioactive agents (or any combinations thereof). The connector may include holes for egress of the flowable material. In some embodiments, the passageway may include a hollow tube extending through the anchorable members and/or the connector, and the hollow tube may also contain holes for egress of flowable material, or may be rupturable to release flowable material.

The system may also include a delivery device for delivering the fracture-stabilization device into the bone in the collapsed (unexpanded) state and for delivery or release of the device within the fracture region and attachment. Because the fracture-stabilization device is at least partially self-expanding, and may be biased into an expanded (anchoring) state, the delivery device may apply force to maintain the fracture-stabilization device in a delivery (collapsed) configuration. For example, a delivery device may include one or more rods. These rods may be configured to releasably engage one or both of the anchorable members. For example, the distal anchorable member may include an attachment site at its distal end configured to releasably attach to a delivery device. In some variations the other (proximal) member includes a second attachment site that can be releasable attached to another portion of the delivery device. The delivery device may therefore apply force to keep the fracture-stabilization device in the collapsed (delivery) configuration. In variations in which the components expandable members and/or connector of the fracture-stabilization device are delivered separately, each component portion may include attachment sites at either end to maintain the delivery configuration.

The connector and at least one of the first or second anchorable members may be threadably connected such that rotation of one of the anchorable members (or the connector) changes the distance between the two anchorable members. Thus, the relative spacing of the members may be adjusted (e.g., by rotating). In some variations, the connector is adapted to modify the length between the anchoring members. The spacing may be increased or decreased. The spacing may be modified either during implantation (in the contracted state) or after implantation (in the expanded state).

In general, the delivery device may be configured to position the first anchorable member, the connector, and the second anchorable member into a bone fracture site. The delivery device may be configured to releasably attach to one end of first anchorable member. In some embodiments that include a delivery device, the device includes a rod that is configured to engage the fracture-stabilization device (or a component of the device) at some location distally from the first end. For example, the rod may extend distally from the delivery device into the continuous passageway, and the rod may be configured to attach to the distal end of the first anchorable member, an end of the connector, or either end of a second member. The delivery device may separately apply force to maintain each expandable member in a collapsed configuration. For example, parallel or telescoping rods may extend in the central passage and attach to various components to apply force sufficient to keep individual members in the collapsed (delivery) configuration or to provide force to expand either or both members of the fracture-stabilization device.

A delivery device for a fracture-stabilization system may also include a sleeve or cannula that encloses (e.g., at least partially radially surrounds) the first anchorable member, the connector, and the second anchorable member. A sleeved delivery device may also include a push (or push/pull) rod configured to extend distally from the applicator to the proximal end of the second anchorable member.

Any of the delivery devices described herein may also be configured to allow removal or readjustment of the fracture-stabilization devices. For example, the connectors between the fracture-stabilization device and the delivery device (e.g., rods, sleeves, etc.) may be reengaged so that the device can be partially collapsed and adjusted or removed.

Also described herein are methods for stabilizing a fractured bone using a fracture-stabilization device. For example, a method of stabilizing a fracture bone may include: forming a passage in the fractured bone through a proximal bone region, across the fracture, and into a distal bone region; positioning a bone fracture-stabilizing system having a first expandable member a connector and a second expandable member in the passage; and anchoring the first anchoring member within the distal bone region and the second anchoring member within the proximal bone region. In some embodiments of the method, the method begins with aligning the proximal bone region and the distal bone region prior to forming the passage in the bone.

The method may also include inserting the anchorable members of the fracture-stabilization device into the passage in a constrained configuration. The anchoring members may be released from the constrained configuration to expand and anchor. For example a fracture-stabilization device may be anchored by detaching the first anchoring member from a rod of the delivery device.

The method may include radially expanding a plurality of bowed struts from each anchorable member to anchor the member within the bone. In some of these embodiments, the struts radially self-expand. Struts may be expanded with a mechanical assist after self-expanding. In some embodiments, the first and second anchorable members expand simultaneously. Alternatively, the first anchorable member expands before the second anchorable member, or vice-versa.

The method may also include cutting the bone as the device expands. For example, the method may include the step of exposing cutting surfaces on the struts as they expand into the anchoring configuration.

In some embodiments, the method also includes applying a flowable material through the continuous passageway. The flowable material may exit the passageway into the surrounding bone. For example, the flowable material may exit openings in the passageway of the first and second member, and/or the connector, flowing a material through the continuous passageway so that at least some material exits holes from the connector. The flowable material may be hardened (e.g. by setting, curing, or otherwise) to form a solid material.

The method of stabilizing a bone fracture may also include the step of altering the distance between the first and second anchoring members. For example, the connector may be rotated to change the distance between the first and second anchoring members.

### BRIEF DESCRIPTION OF THE FIGURES

FIGS. 1A-1F show a fracture-stabilization device with a circular cross-section having two expandable members, each with four radially expandable struts. The struts have a flat expanding surface. FIG. 1A is a perspective view of the body of the device. FIG. 1B is a side view of the body of the device showing slots forming the struts. FIG. 1C is a cross-sectional view of the device. FIG. 1D is a perspective view of the device after the struts have radially expanded. FIG. 1E is a side view of the device after the struts have radially expanded. FIG. 1F is an end view of the device after the struts have radially expanded.

FIGS. 2A-2F show an internal-external, or double-bodied, fracture-stabilization device, wherein each body includes two expandable members (or regions), each with four expandable struts. The struts of the internal and external bodies are staggered with respect to each other. FIG. 2A is a perspective view of the device in the unexpanded (insertion) configuration. FIG. 2B is a side view of the body of the device. FIG. 2C is a cross-sectional view of the device. FIG. 2D is a perspective view of the device after the struts have radially expanded. FIG. 2E is a side view of the device after the struts have radially expanded. FIG. 2F is an end view of the device after the struts have radially expanded.

FIGS. 3A-3F show a fracture-stabilization device with a rectangular body and four radially expandable struts, each arising from a cut through a flat surface of the body and expanding with a leading sharp edge. FIG. 3A is a perspective view of the body of the device. FIG. 3B is a side view of the body of the device showing slots forming the struts. FIG. 3C is a cross-sectional view of the device. FIG. 3D is a perspective view of the device after the struts have radially expanded. FIG. 3E is a side view of the device after the struts have radially expanded. FIG. 3F is an end view of the device after the struts have radially expanded.

FIGS. 4A-4F shows a fracture-stabilization device with a rectangular body and two radially expandable struts arising from length-wise cuts in a flat surface of the body and expanding with a leading flat edge. FIG. 4A is a perspective view of the body of the device. FIG. 4B is a side view of the body of the device showing slots. FIG. 4C is a cross-sectional view of the device. FIG. 4D is a perspective view of the device after the struts have radially expanded. FIG. 4E is a side view of the device after the struts have radially expanded. FIG. 4F is an end view of the device after the struts have radially expanded.

FIGS. 5A-5F show a fracture-stabilization device with a rectangular body and two radially expandable struts formed by length-wise cuts at a vertex of the rectangle, each strut expanding with a leading sharp edge. FIG. 5A is a perspective view of the body of the device. FIG. 5B is a side view of the body of the device showing slots to be cut from which struts will emerge. FIG. 5C is a cross-sectional view of the device. FIG. 5D is a perspective view of the device after the struts have radially expanded. FIG. 5E is a side view of the device after the struts have radially expanded. FIG. 5F is an end view of the device after the struts have radially expanded.

FIG. 6 shows a single anchorable member with two radially opposed struts in an expanded configuration, the member being a component joinable with a connector portion and a second anchor to form a complete fracture fixation device.

FIG. 7 shows a perspective view of a single anchorable member with three radially distributed struts in an expanded configuration, the member being a component that is joinable with a connector portion and a second anchor to form a complete fracture fixation device.

FIG. 8 shows a perspective view of a single anchorable member with four radially opposed struts in an expanded configuration, the member being a component joinable with a connector portion and a second anchor to form a complete fracture fixation device, the anchorable member further including a central rod that maintains a continuous passageway with a connector in the fully assembled device. The connector portion and/or rod include holes from which a flowable bone cement may be ejected.

FIGS. 9A and 9B show a fracture-stabilization device with a rectangular body and two radially expandable struts emanating from length-wise cuts at a vertex of the rectangle. This device is similar to that depicted in FIG. 5 except that the corners of the rectangle have been pinched or crimped in, giving the corner an angle more acute than 90 degrees. These acute corners become the leading edge of a strut as it expands, and in this embodiment the leading edge is particularly sharp. FIG. 9A is a perspective view of the body of the device. FIG. 9B is a partial view through an expanded struts.

FIGS. 10A-10F show one anchorable member of an embodiment of a fracture fixation device with a linearly corrugated surface, from which nine expandable struts emanate. FIG. 10A shows the body of the anchorable member in a linearly constrained, non-radially expanded configuration. Slots are present though not visible in the inner vertex of corrugations. FIG. 10B shows expansion of the expandable struts to a first position, which may either be a partial or fully self-expanded configuration. FIG. 10C shows expansion of the expandable struts to a second position, more expanded than the first position of FIG. 10B. FIG. 10D shows a linearly cross sectional view at position 10D of FIG. 10A, showing the corrugated nature of the body of the expandable member. FIG. 10E shows a linearly cross sectional view at position 10E of FIG. 10B, showing the M-shaped cross-sectional profile the expanded struts. FIG. 10F shows a linearly cross sectional view at position 10F of FIG. 10C, showing the flattened M-shaped cross-sectional profile the expanded struts.

FIG. 11A shows a fracture-stabilization device exploded into three parts, illustrating various dimensions of the device. FIG. 11B shows a cross section of the body of an anchorable member. FIG. 11C shows a cross section of the struts at their most expanded point. FIG. 11D shows a cross section of an alternative embodiment with three struts rather than four struts.

FIGS. 12A-12E illustrate deployment of a fracture-stabilization device into a hip bone, passing through a point just below the greater trochanter of the femur, across the fracture, and into the head of the femur. FIG. 12A shows a drill bit forming a passageway for the device. FIG. 12B shows the formed passageway prepared to receive the device. FIG. 12C shows a delivery device inserted into the passageway, positioning the distal anchorable member of a device. FIG. 12D shows the first or distal anchorable member after expansion of the struts. FIG. 12E shows the device in situ, the proximal or second expansion member after expansion of its struts, and after the two anchorable members of the device have been drawn together, tightening the fracture zone.

FIG. 13 depicts two fracture-stabilization devices implanted into a fracture of a hip in location similar to that depicted in FIG. 12.

FIG. 14 depicts two fracture-stabilization devices implanted into a flat bone such as a skull plate, the devices substantially flat in their expanded profile, the expandable members having two struts.

FIG. 15 shows one variations of a fracture-stabilization kit, the kit including an Allen head tool, a first and a second anchorable member, a connector, a delivery device, a container of flowable cement, a push rod for delivering a distal anchor, and a delivery rod for delivering a proximal anchor.

FIGS. 16A-16O illustrate a fracture fixation device in three conjoinable units (first and second anchorable members and a connector portion), as well as anchoring opposition rods, in various stages of assembly, but ultimately into a complete and implanted device. The device is shown variously both in isolation, ex situ, and as implanted, in situ. FIG. 16A shows the first anchorable member constrained in a linear configuration by a portion of the inserter, the anchorable member threadably-connected to the delivery device.

FIG. 16B shows the first anchorable member after the inserter (push or opposition rod portion) has been partially withdrawn, allowing the anchorable member to self-expand.

FIG. 16C shows the first anchorable member being further expanded by a mechanical assist, the opposition rod remaining engaged at the distal portion of the first expandable member, and being pulled proximally by the rod, which is still engaged at the distal end of the first anchorable member. This is an optional step; FIG. 16D continues as if this step had not been taken.

FIG. 16D shows the first anchorable member released from the linearly constraining opposition rod and in a self-expanded configuration, the rod now withdrawn from the first anchorable member.

FIG. 16E shows the first anchorable member in situ in the self-expanded configuration, as it's anchored in its expanded configuration.

FIG. 16F is an in situ view showing the connector portion being threadably connected to the first anchorable member, the connector being deployed by a delivery device, the delivery device threadably connecting the connector to the first anchorable member.

FIG. 16G shows first anchorable member and the connector now conjoined, and a second anchorable member now being brought into position to engage the connector portion by a delivery device with a rod constraining the anchorable member in its linear configuration.

FIG. 16H shows the second anchorable member now in contact with the connector, the member still being linearly constrained by the rod of the delivery device.

FIG. 16I shows the second anchorable member now released from its proximal attachment to the rod, and the anchorable member linearly contracted and radially expanded.

FIG. 16J is an in situ view showing the second anchorable member implanted and expanded, and the deploying rod now withdrawn.

FIG. 16K shows an Allen wrench connector deployer extending through the second anchorable member to engage the connector and beginning to rotate the connector with respect to the two anchorable members.

FIG. 16L shows the first and second anchorable members now drawn together by a turn-buckle rotation of the connector threadably engaged with both the first and second anchorable members.

FIG. 16M is an in situ view showing the fully assembled device in its anchoring configuration, the two anchorable members drawn together to the desired degree toward the connector, and the deployment device having been withdrawn.

FIG. 16N shows an injector tube inserted into the device and a flowable cementing composition being injected through the passageway extending there through, the cement composition being emitted into the space within the expanded struts of the anchoring members, and through holes in the connector to emerge into available space peripheral to the connector.

FIG. 16O shows the device, the cement inserter removed, the device now fully implanted, and stabilized by the cement now hardened.

FIGS. 17A-17E show various embodiments of fracture fixation devices that have dissimilar first and second anchoring or anchorable members for custom fitting into fracture sites. FIG. 17A is a device with a three-strut anchorable member and a two-strut anchorable member, in each case that struts curvilinear and asymmetrically bowed. FIG. 17B is a device with a two-strut anchorable member and a four-strut anchorable member, in case the struts are symmetrically bowed and having substantially straight segments. FIG. 17C is a device with a four-strut anchorable member that is significantly larger than its two-strut companion. FIG. 17D shows a device with three anchorable members, each member having two struts, the members expanding in different radial orientations, two with substantially straight segments in the struts, and a third with curvilinear struts. FIG. 17E is a device with an anchorable member having two asymmetrically bowed struts and a central hollow rod and a second anchorable member with four symmetrically bowed struts and without a central rod.

### DETAILED DESCRIPTION OF THE INVENTION

Described herein are bone fracture fixation systems and devices, and methods of using them to repair fractures. The figures illustrate various embodiments of the system. Although the description specifies the use of embodiments of the fracture fixation system to repair a fracture of the femoral head, the devices, systems and methods described herein may be used to repair other fractures as well. Embodiments of the devices and methods provided herein may be applied to a wide variety of bones and to various fractures that they may incur. Sizes and specifics of device conformation and configuration are readily varied, and devices may be assembled so as to fit the specifics of a particular fracture site. Further, the devices may be applied to regions of bone that include cancellous bone, cortical bone, or both types of bone.

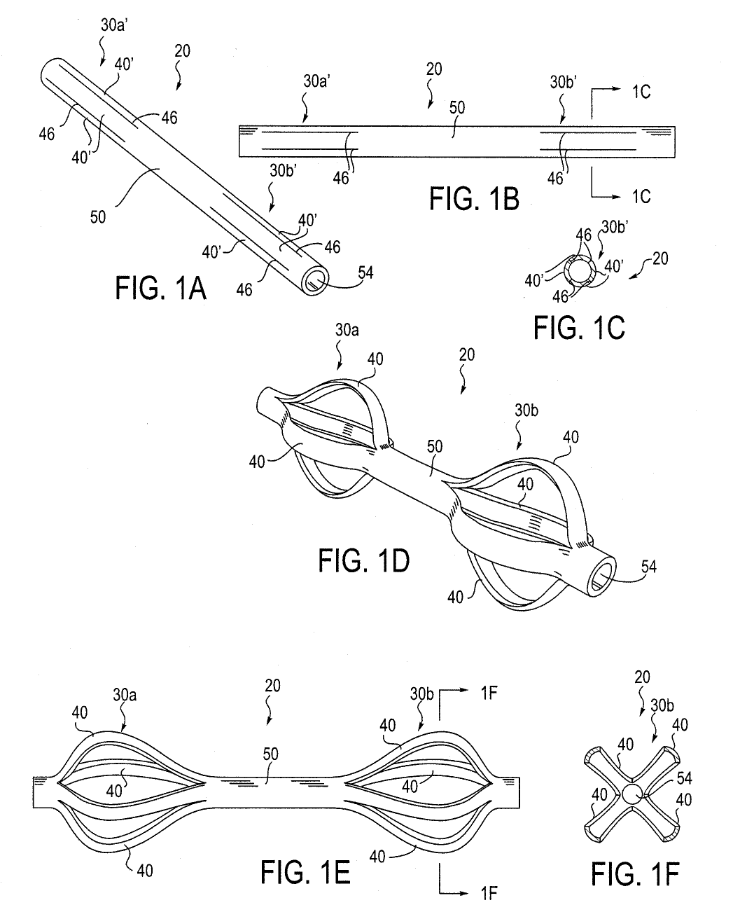

In general, the fracture-fixation devices described herein include two anchorable (or anchoring) members connectable or connected by a connector. These anchorable members typically include expanding (e.g., self-expanding) structures such as struts. As will be seen, struts may be highly variable in form, and may include for example, outwardly expanding structures the lead with flat, rounded, or sharp cutting edges. In some fracture sites, a cutting edge may be preferred as a way to cut into the bone most effectively to form an anchor, and in other sites, it may be preferred to lead with a flat of rounded surface that can provide more substantial outward support to a bone when the device is in its final anchoring position. Various embodiments and features of the devices, system and method will be described with general references to FIGS. 1-17, and FIGS. 1-17 will be detailed individually in greater detail thereafter.

A system for stabilizing or fixing a bone fracture 20 may include two anchorable members 30 with an intervening connector piece 50. Anchorable members 30 can also be referred to as a first member 30a and second member 30b. Typically, the first member 30a is distal with respect to the second or thus proximal member 30b, distal referring to a position furthest from the delivery device (e.g., deepest within a fracture site from the perspective of a physician implanting the device) or from the perspective of a delivery (or deployment) device that positions the device within the site of fracture. The anchorable members typically have two configurations; one configuration is substantially collapsed, which may be linear in orientation. This is the non-anchoring (or delivery) configuration of the member in which it may be deployed and positioned in a fracture site. The second configuration is an anchoring (or expanded) configuration, which typically includes a radially expanded structure. An anchorable member in a constrained or non-expanded configuration may be labeled as member 30′ (30 prime).

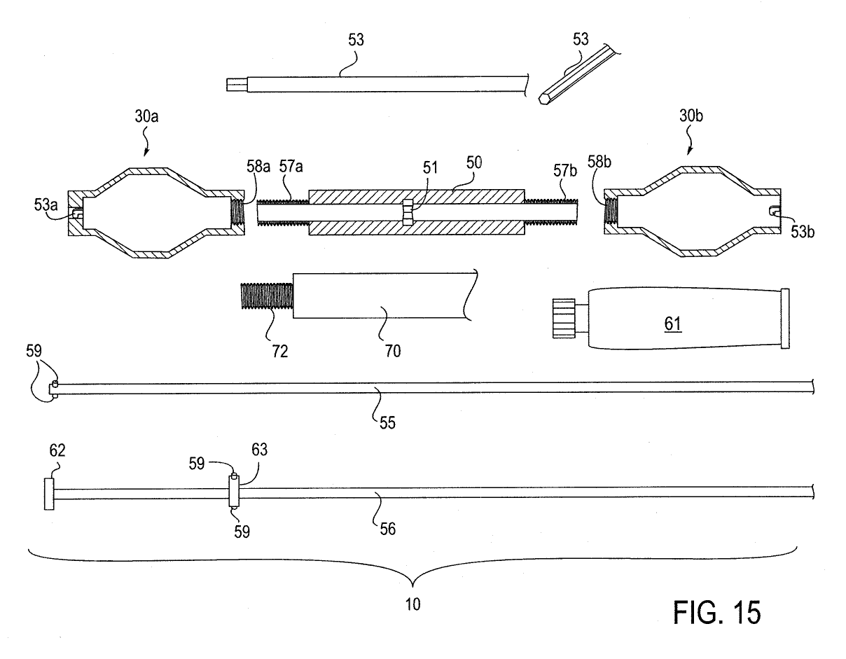

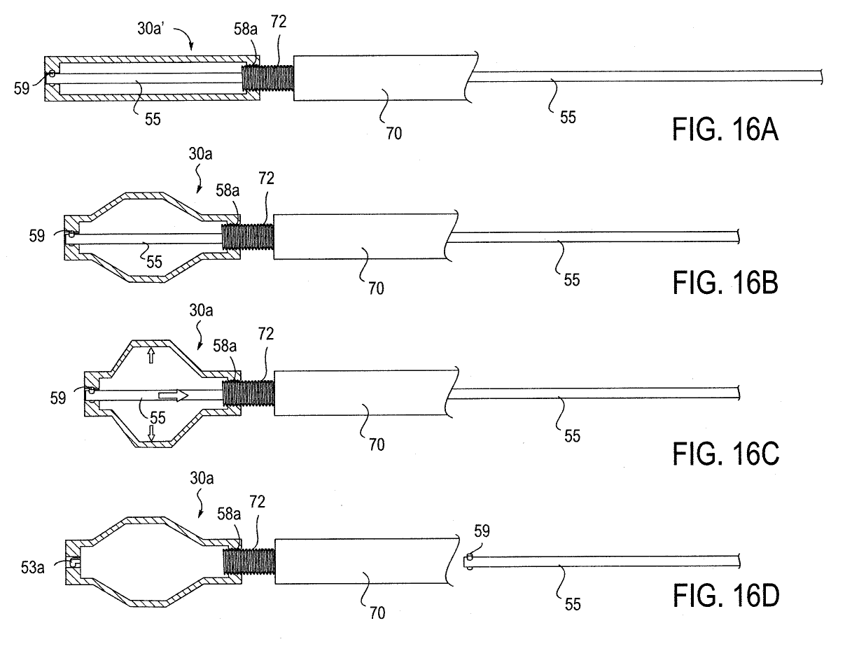

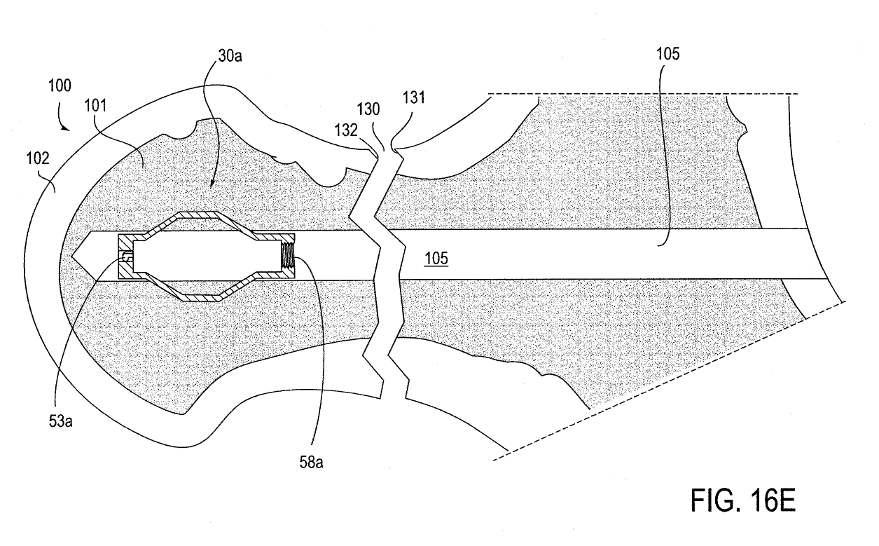

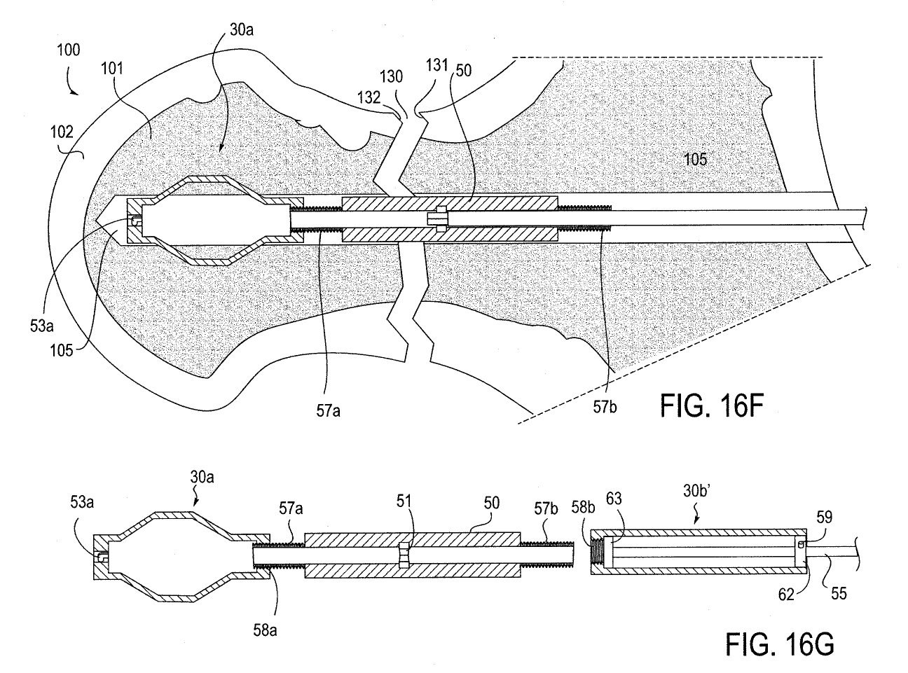

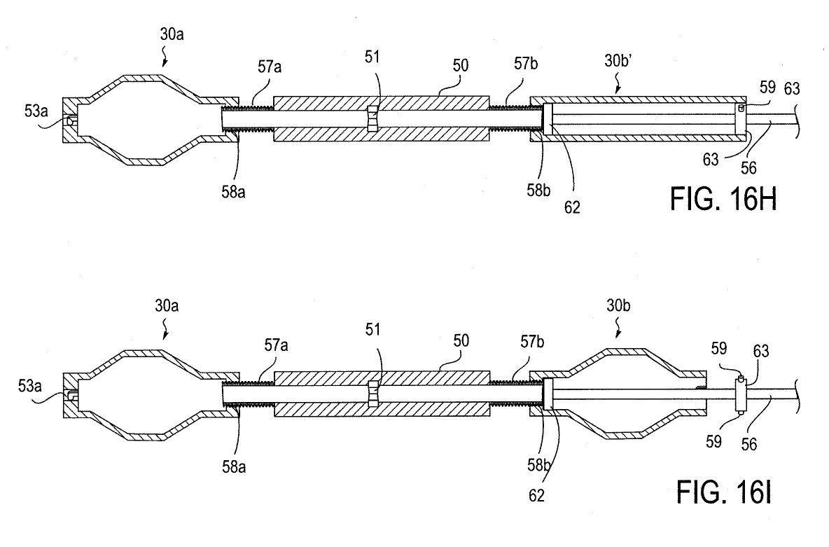

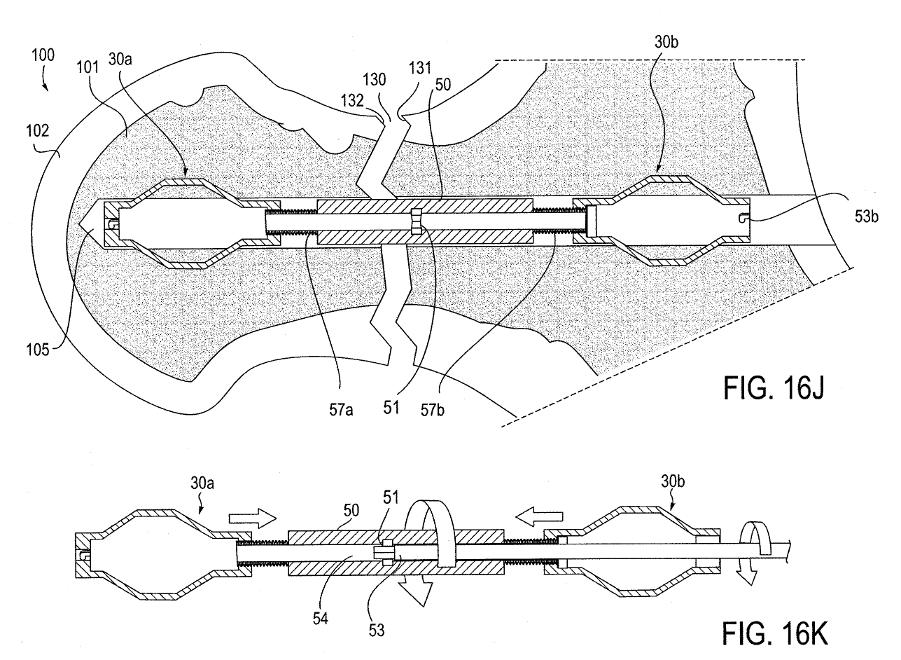

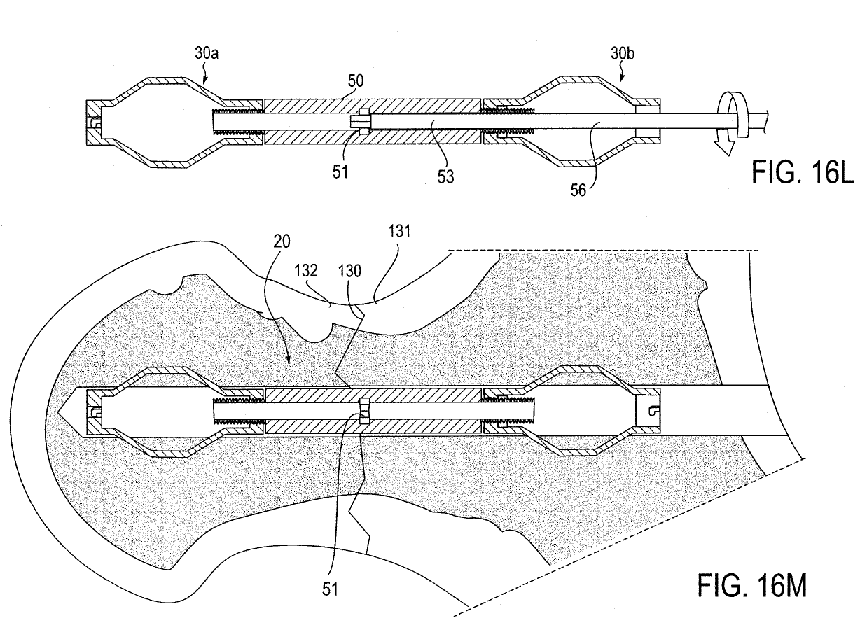

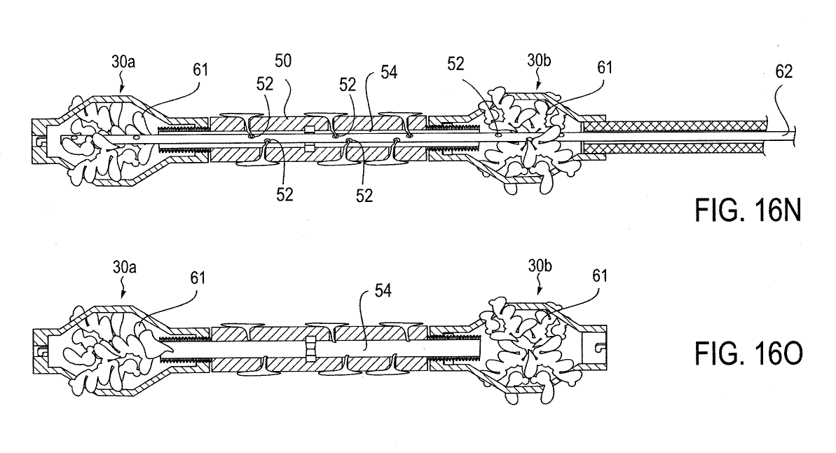

An assembled fracture stabilizing device may be formed in various ways. In some embodiments of device 20, two anchorable members 30 and a connector piece 50 are fabricated as a single integrated unit. In other embodiments, a proximal anchorable member 30b and a connector 50 are conjoined into a single integrated unit, and a distal anchorable member 30a is a separate piece that is joinable with the integrated proximal anchor 30b and connector. In other embodiments, a distal anchorable member 30a and a connector 50 are conjoined into a single integrated unit, and a proximal anchorable member 30b is a separate piece that is joinable with the integrated distal anchor and connector. In still other embodiments, a first or distal anchorable member 30a, a connector 50, and a second or proximal anchorable member 30b are all separate pieces that are conjoinable. In some embodiments of the fracture fixation device, the invention includes a kit of parts that may be assembled into a complete device 20 before implantation in a fracture site, or such parts may not be fully assembled until the time when they are being positioned within the fracture site. See FIG. 15 for an embodiment of a kit of parts. FIGS. 16A-16O illustrate one variation of a method of inserting a first anchorable member, a connector, and a second anchorable member in order to assemble a complete device. In some variations, a connector is a connector region extending from one or both anchorable members.

In general, when any of the connector and both anchorable members are separate or separable, they may be connected in any appropriate manner. For example, they may be threaded (e.g., connected by screwing), or may be slidably connected (e.g., one or more anchorable members may slide over the connector region) that can interlock.

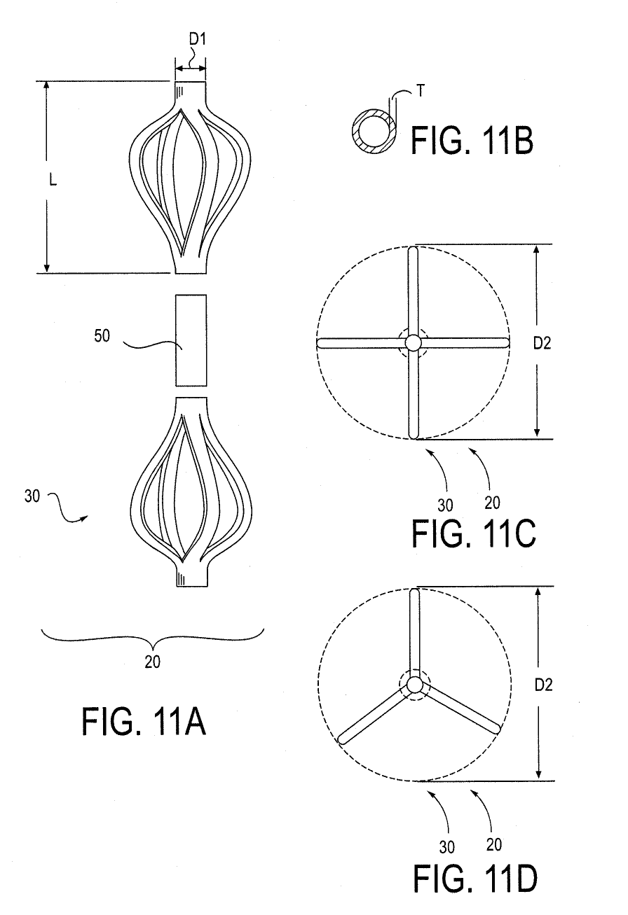

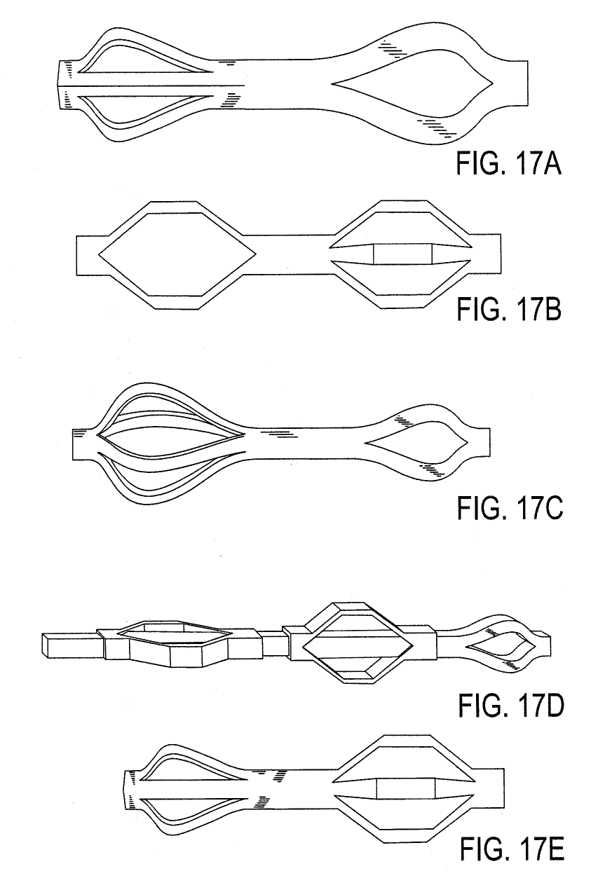

The dimensions of anchorable members 30 of a fracture stabilizing device 20 may be selected according to their intended site of use. The exemplary dimensions provided here are to help in providing an understanding, and are not intended to be limiting. FIGS. 11A-11D show an embodiment of the device 20 and provides visual reference for various dimensions, and is described in further detail below. As noted above, the fracture fixation device 20 may be embodied as a kit of parts. These parts may have a modular character in that, in spite variations in size and form of some regions, there may be limited variation in some dimensions. For example, the diameter of the body may have a limited number of sizes so that parts are readily conjoinable around common features, particularly points of threadable connections, as between a connector and anchorable members, and as in the size of the lumen extending through a connector and as such lumen or rod may further extend through anchorable members. A device 20 assembled from various parts could have identical first and second anchorable members, or the members could be dissimilar. The great variety of devices that may be generated from such a system allows for custom fitting of a device to the dimensions of a fracture and the surround fracture regions; a few such exemplary devices with dissimilar first and second anchorable members are depicted in FIGS. 17A-17E.

Anchorable members 30 (and possibly connector 50) may be formed from any appropriate material. In particular, shape-memory materials. Anchorable members may be formed by “prebiasing” them into a shape such as an expanded (anchoring) shape. In some variations, components of the fracture-fixation device are formed at least partially from a resiliently deformable material such as a plastic, metal, or metal alloy, stainless steel, for example, or a shape memory (and super-elastic) metal alloy such as Nitinol. A detailed description of materials that may be suitable for the fabrication of the present fracture fixation device may be found in U.S. patent application Ser. No. 11/468,759, which is incorporated by this reference in its entirety. In typical embodiments of an anchorable member, the preferred state of the member is that of the radially-expanded anchoring configuration. In these embodiments, the unexpanded configuration that is appropriate for deployment and initial positioning within a fracture site is a constrained configuration.

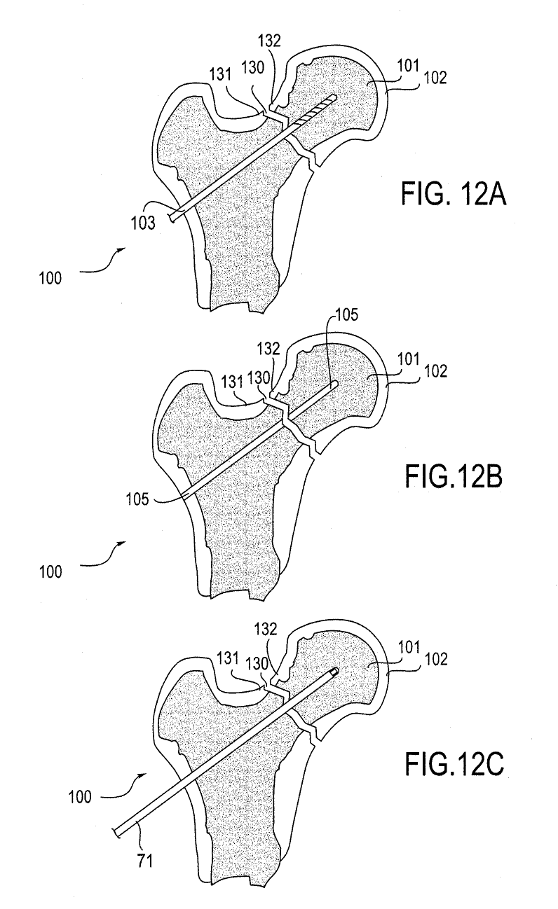

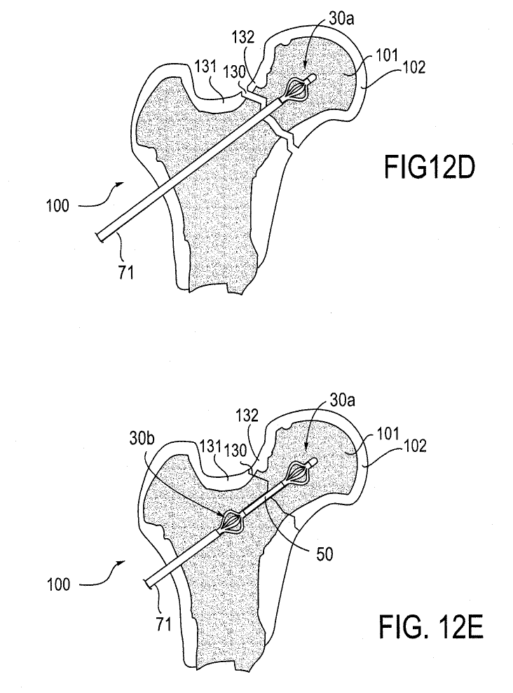

Embodiments of the invention may constrain an anchorable member 30′ in at least two ways, which will be described in greater detail below. Briefly, one approach is that of confining the member within an enclosing cannula or sleeve 71 that physically prevents radial expansion. A delivery device including a cannula or sleeve is shown in FIGS. 12A-12 E, wherein a fracture fixation device configured as a single conjoined unit prior to delivery is implanted in a fracture zone of a femoral head. In these embodiments, the delivery device may include a push rod, to distally eject a fracture fixation device. In some variations, the device, or regions of the device (e.g., the anchorable members) are place under tension by the delivery device to prevent them from expanding. Radial expansion may shorten or contract the anchorable members of device. Thus, a delivery device may include one or more attachment sites to constrain the anchorable members from expanding. For example, a delivery device may apply tension to the anchorable members through a rod (e.g., a length-constrainment rod) extending distally from a delivery or deployment device 70. The rod may prevent shortening of length and radial expansion of anchorable members. The rod may be slidable within the delivery device, but can be held (e.g., locked) in an extended position to prevent deployment of the anchorable member. An example of a delivery device including a rod for applying or maintain tension is depicted in FIGS. 15 and 16A-16O, and described in considerable detail further below.

An anchorable device 20 may include two anchorable members 30 and a connector 50, and each of these components includes a passageway or channel 54 there through, that forms a continuous passageway 54 though the fracture-fixation device. The passageway 54 may form a lumen through which a rod 57 may be inserted, and through which a flowable cementing or bone-filling material 61 may be conveyed. The passageway may also be a hollow tube 54 that may form a strengthening structural element for the device 20 as a whole. In some embodiments, only the connector portion includes hollow tube 54; in other embodiments, the hollow tube is included as a structural feature of one or more of the anchorable members. The connector and/or tube 54 also may also be configured so that the anchorable members 30 may be moved closer or further apart from each other. For example, the connector and/or tube may be threaded and rotation of either the connector or one or more of the anchorable members may draw the members closer together.

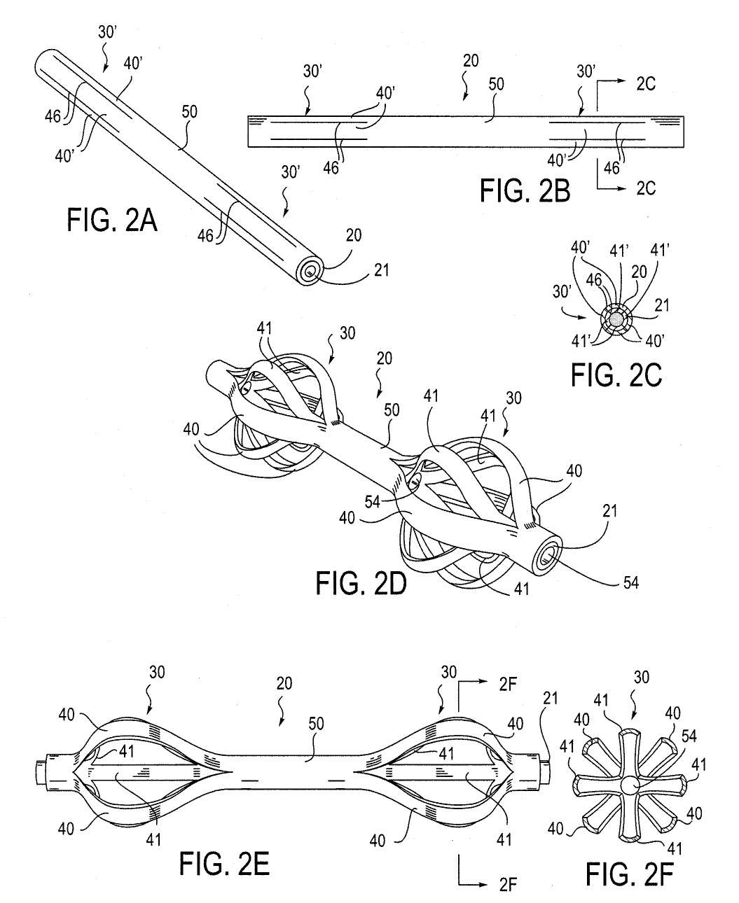

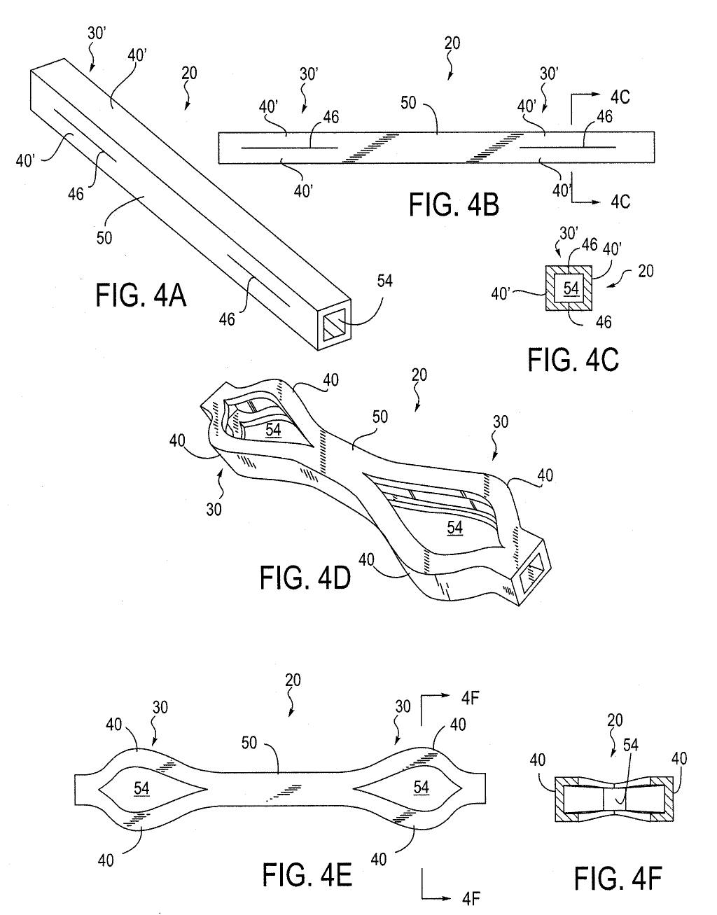

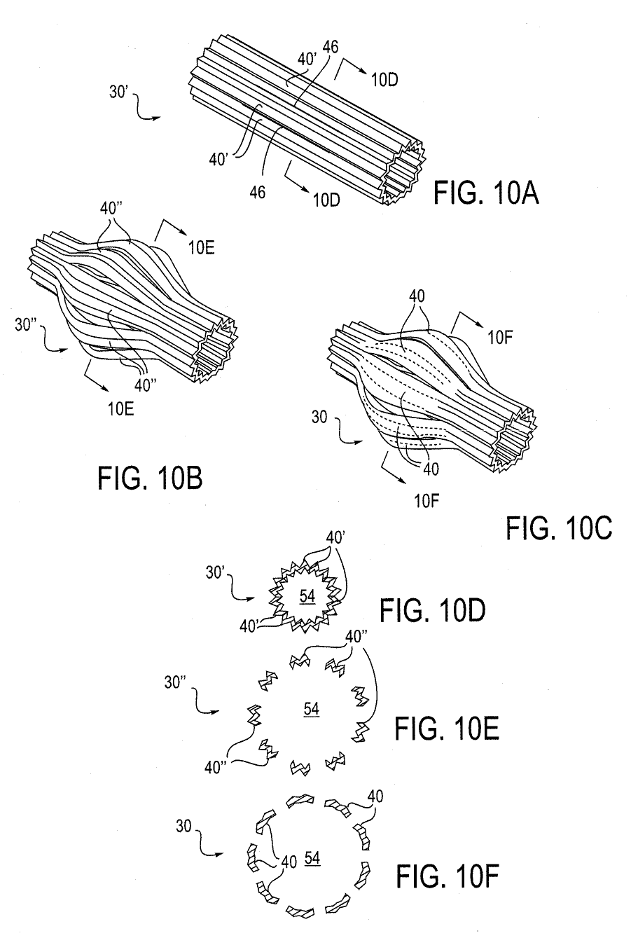

In its constrained (delivery) configuration, an anchorable member 30′ may be in the form of a substantially hollow tube. In some variations, the cross-section of the fracture fixation device is substantially circular or oval (as in FIGS. 1 and 2), particularly the expandable members. In some variations, it is a sided-structure, e.g., having three sides, four sides, or more than four sides (as in FIGS. 4, 5, and 9). In an embodiment with four sides, a rectangular configuration may have four sides of equal length. Further variations of the cross sectional profile occur in other embodiments. For example, the vertices or corners of a sided-embodiment may be pinched or crimped in (FIGS. 9A and 9B), this configuration may create a more acute cutting edge on the struts as they undergo their self-expansion upon release of the device from constraint. In other embodiments, the surface may be substantially round in profile, but embellished with linear corrugation, as show in FIGS. 10A-10C. In this configuration, the linear folds of the struts may impart strength to the struts that remains even in the expanded configuration of the struts.

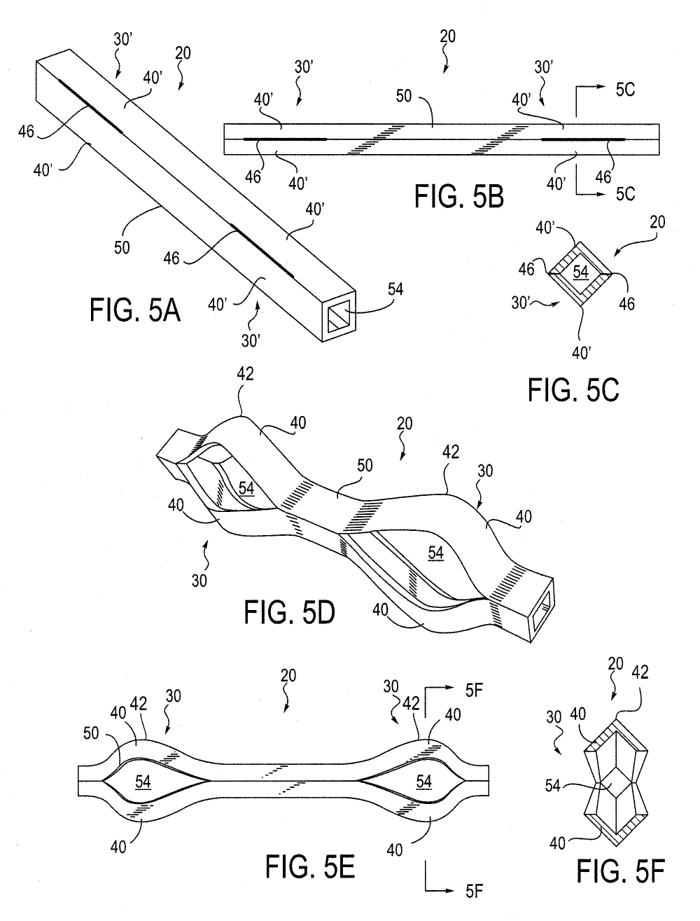

As described in U.S. patent application Ser. No. 11/468,759 (Pub No. US 2007/0067034 A1) and U.S. Provisional Patent Application No. 60/916,731, slots or slits 46 may be cut lengthwise in a tube to form nascent struts 40. With metallurgical methods well known in the art such as heat treatment, the struts 46 may be configured into a preferred configuration such as a bow. In some device embodiments, the configuration of bowed struts may be linearly symmetrical or substantially symmetrical (as shown in FIGS. 5, 10, and 15), and in other embodiments, the bow may be asymmetrical (as shown in FIGS. 1-4), with the maximal expanded portion skewed either toward the distal or proximal end of an anchorable member. Other configurations of symmetrical and asymmetrical struts may also be used.

An anchorable member 30 having three struts comprising the body 45 of device 20 typically has a triangular cross section, the struts formed by slots cut through the surface of each of the three sides. In an embodiment where the triangle of the cross-section is equilateral, the struts are radially distributed equally from each other, with 120 degrees separating them (see alternative embodiment in FIG. 11). In other embodiments, where the triangle of the cross sections is not an equilateral triangle, the radial angles of struts may include two that are equal, and a third angle that is not equal to the other two. There may be some benefits associated with anchorable member embodiments with three struts compared with four or more struts. The struts formed are wider, and thereby may be stronger than members having four struts emanating from a device body of the same diameter.

In some four-strut variations, the body 45 of the device 20 is either square or circular in cross section, and the four struts 40 emanating from the body are typically equally spaced apart at 90 degrees, or they may be radially distributed such that the angles formed include two angles greater than 90 degrees and two angles less than 90 degrees. A body 45 with a square cross section typically is appropriate to support struts that are spaced apart by 90 degrees, the strut-forming slots positioned centrally lengthwise along the body (FIGS. 4A-4F). This configuration also imparts a 90 degree leading edge on struts 40 formed therefrom, such an edge being useful in cutting through bone. In many embodiments of the invention, efficiency in cutting through bone, either or both cortical bone or cancellous bone, is advantageous. Cutting may separate bone mass to allow strut movement through bone with minimal compression of bone, and thus minimal disturbance of bone tissue in regions adjacent to the path of separation. Bone (particularly cortical bone) may be cut only slightly, and may serve to help anchor the device in or to the bone. In other embodiments, it may be desirable that struts 40 have a surface that presents a flat face for bone support, e.g., expandable members having a circular cross section (as illustrated in FIGS. 1A-1F and 2A-2F).

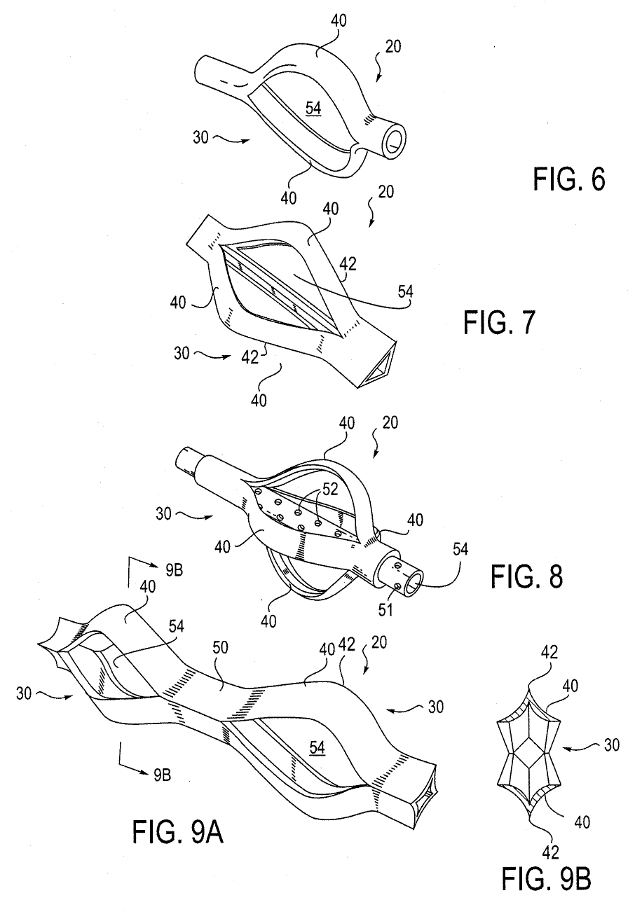

In some variations, the anchorable member includes only two struts. In these variations, the 45 of a device 30 may be circular (FIG. 6) or square (FIGS. 4A-4F) in cross section. In embodiments having a square cross section, lengthwise slots 46 may be made at opposite vertices of the square, in which case the two struts formed therefrom have a 90 degree leading edge (FIGS. 5A-5F). For example, a body having a circular cross section may include lengthwise slots 46 that may be made at radially opposite positions, in which case the two struts formed therefrom have a broad leading edge (FIG. 6). In some embodiments of a fracture-fixation device 20, a broad leading edge may be beneficial if the leading edge is intended to provide support to a bone surface from within.

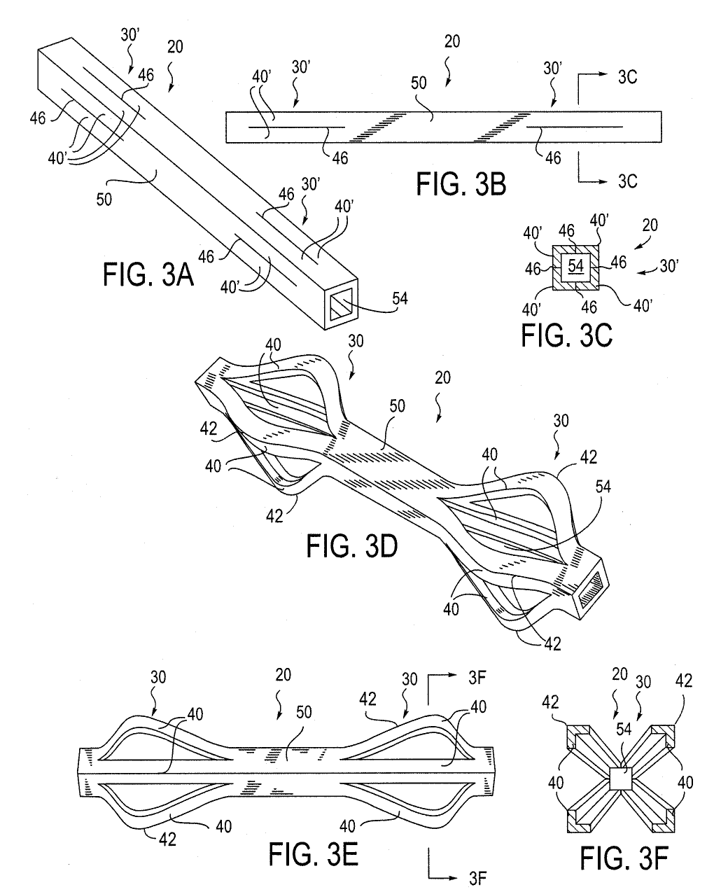

As mentioned, the struts 40 may be formed by cuts or slots 46 in the body of the device 20 and may include a sharp cutting edge 42 useful for cutting, scoring or securing to bone (either cancellous bone 101 or cortical bone 102) as the struts radially expands upon being released from constraint (FIGS. 3A-3F, 5A-5F, and 9A-9B). A sharp edge may be derived from a vertex or corner of the device body as seen in cross section. Thus, for example, a rectangular body or a triangular body can generate struts with a sharp leading edge as the struts expand. In typical embodiments, for example, where struts are formed from a the body of an anchorable device with a rectangular cross section, cuts in the metal to create slots are made in the central portion of sides of the rectangle, and struts 40 are formed at the vertices of the rectangle. Thus, in some embodiments, the cutting edge 42 of a strut 40 may have a leading angle of about 90 degrees. In other embodiments of an anchorable member 30 with a rectangular cross sectional profile, the vertices of the rectangle may be crimped or pinched in order to create corner angles that are more acute than 90 degrees (FIGS. 9A and 9B). In embodiments such as these, the cutting edge 42 or a strut 40 may have a leading angle more acute than 90 degrees. In embodiments of an anchorable member 30 with an (equilateral) triangular cross section, the vertices of the triangle have an angle of 60 degrees, and thus struts 40 formed from such vertices have a cutting edge 42 with an angle of 60 degrees.

In some embodiments of a fracture fixation device 20, the first anchorable member 30a and the second anchorable member 30b are identical (e.g., FIGS. 1-6). In other embodiments of a fracture fixation device 20, the first 30a and second 30b anchorable members are dissimilar (FIGS. 17A-17E). Fracture-fixation device may include anchorable members that are different in size (e.g., length of body 45, length of struts 40, differences in diameter of the body 45), different in the radial expansiveness of the released configuration of struts 40, different with regard to the symmetry or asymmetry of bowed struts 40, or different in any other anchorable member parameter. By such variations in form of the two anchorable members 30, a fracture fixation device 20 may be tailored to suit the particular dimensions of a target fracture site. As described above, a device 20 may be further tailored or fitted to a target fracture site by any of the variations in size provided by embodiments of anchorable members 30 and their components, such as struts 40 or connector 50.

Some embodiments of a fracture fixation device 20 may include an internal anchorable member within an external anchorable member (FIGS. 2A-2F). The benefit provided by this general configuration is that it provides more surface area (e.g., twice as much) for anchoring within a given anchoring volume of bone than does a single anchoring member. Typically, the number of struts in the companion internal and external bodies are the same, and are radially staggered with respect to each other, so that the struts of the inner body may emerge in the spaces between the struts of the outer body. The struts of the inner and outer bodies may be of about the same length and bowed outwardly to about the same degree, as they are in FIGS. 2A-2F. In other embodiments, the struts of the inner body may be shorter in length, or bowed outward to a lesser degree than the struts of the outer body.

A fracture stabilizing system 10 may included one or more delivery devices. By way of example, a delivery device may be a sleeve or cannula 71 which constrains embodiments of device 20 for deployment (FIGS. 12A-12E). Deployment occurs by means of a push rod extending distally in the delivery device to a point of contact on the proximal surface of the second or proximal anchorable member 30b. By pushing the device 20 distally and at the same time withdrawing the cannula from an implantation site, the first or distal anchorable member 30a emerges from the cannula and self-expands as it is released from the lateral or circumferential constraints of the cannula. As the cannula is withdrawn further in the proximal direction from an implantation site and simultaneously continuing to push the device distally out of the cannula, a connector portion 50 and a second or proximal anchorable member 30a emerge in sequence. As the second anchorable member is released from the circumferential constraints of the cannula, it self-expands, as did the first anchorable member.

A second exemplary delivery device 70 illustrated herein generally constrains the fracture-fixation device to a linear configuration and prevents expansion of struts by applying tension across at least a portion of the device to prevent contraction of shortening of the body of the device (as in FIGS. 16A-16O). Embodiments of this delivery device may be similar to embodiments of delivery devices disclosed in detail in U.S. Provisional Patent Application No. 60/906,731, filed on May 8, 2007, and which is hereby incorporated in its entirety.

A fracture-fixation device may be delivered by providing a delivery device that constrains the anchorable members from contraction. The delivery device can be used to sequentially expand a first anchorable member, and a second anchorable member, either sequentially or simultaneously. The device may be inserted with all of the components of the fracture-fixation device attached (e.g., fully assembled) or with them in components that are joined after (or during) delivery.

As described above, some embodiments of device 20 may be fabricated from a superelastic shape memory alloy such as Nitinol, in which case struts 40 may be configured to self-expanding when released from constraint in a radially non-expanded (or linear form). When implanted in bone, particularly in hard cortical bone, expansion of struts may be resisted by surrounding bone. Facing such resistance, expandable struts 40 may not expand to their full potential. Inasmuch as greater anchoring stability is associated with full radial expansion, it may be advantageous to mechanically assist struts in their expansion. Additional mechanical expansion may be achieved by drawing the distal and proximal ends of anchorable members closer together. FIG. 16C shows an exemplary mechanism by which mechanical force is applied to partially expanded struts 40 in order to assist in their full expansion.

Following implantation of a fracture fixation device, a flowable bone filling composition or cement 61 such as PMMA (polymethylmethacrylate) may injected into the fracture region through a trocar and cannula system into the passageway 54 of a device 20. There are many suitable materials known in the art for filling in vacant spaces in bone, some of these materials or compositions are biological in origin and some are synthetic, as described in U.S. patent application Ser. No. 11/468,759, which is incorporated by reference herein. From the passageway, the material flows into the open space within the anchorable members and to some degree, into the peripheral area surrounding the device. The flowable cementing material may contain radiopaque material so that when injected under live fluoroscopy, cement localization and leakage can be observed.

Another example of bone cementing material is provided by a ceramic composition including calcium sulfate calcium hydroxyapatite, such as Cerament™, as manufactured by BoneSupport AB (Lund, Sweden). Ceramic compositions provide a dynamic space for bone ingrowth in that over time, they resorb or partially resorb, and as a consequence provide space for ingrowth of new bone. Bioactive agents may also be included in a cementing composition, such as osteogenic or osteoinductive peptides, as well as hormones such at parathyroid hormone (PTH). Bone Morphogenetic Proteins (BMPs) are a prominent example of effective osteoinductive agents, and accordingly, a protein such as recombinant human BMP-2 (rhBMP-2) may included in an injected bone-filling composition. In this particular context, BMPs promote growth of new bone into the regions in the interior of the expanded struts and around the periphery of device 20 in general, to stabilize the device within new bone. A more fundamental benefit provided by the new bone growth, aside from the anchoring of the device 20, is simply the development of new bone which itself promotes healing of a fracture. In some embodiments of the invention, antibiotics may be included, particularly when there is reason to believe that the fracture site may have been infected. With the inclusion of bioactive agents such as bone growth or differentiation factors, or antibiotics or other anti-infective agents, embodiments of the fracture fixation device become more than a fracture stabilizing or fixation device, as such embodiments take on the role of an active therapeutic or drug delivery device. In general, any appropriate flowable material may be injected into the passageway formed through the fracture-fixation device. In some variations the device (e.g., the proximal end of the fracture-fixation device) may be adapted to receive a device for delivering flowable material.

Examples of fracture-fixation devices, system and methods of using them are provided below, including methods of implanting the device across a fracture to stabilize it and to promote its healing, as particularly detailed in FIGS. 1-17.

For example, FIGS. 1A-1F provide views of a fracture-stabilization device 20 with a circular body having a lumen 54 and two anchorable members 30a, 30b, each with four radially expandable struts 40′, the struts having a flat expanding surface, and a connector portion 50. FIG. 1A is a perspective view of the body of the device. FIG. 1B is a side view of the body of the device showing slots 46 to be cut from which struts will emerge. FIG. 1C is a cross-sectional view of the device. FIG. 1D is a perspective view of the device after the struts 40 have radially expanded. FIG. 1E is a side view of the device after the struts have radially expanded. FIG. 1F is an end view of the device after the struts have radially expanded. A number of structural features of embodiments of the dual-anchoring system 20 described herein, such as slots 46, struts 40, and anchorable members in general, as well as methods of delivery and implantation are similar to features of a vertebral body stabilization device with a single anchorable member, as described in U.S. patent application Ser. No. 11/468,759, which is incorporated into this application, and which may help in the understanding of the present invention.

FIGS. 2A-2F provide views of an internal-external, or double-bodied, fracture-stabilization device, the outer body 20 surrounding an internal body 21. Each body has a lumen 54 and two anchorable members 30, each with four expandable struts 40′, the struts 41 of the internal body and the struts 40 of the external body staggered with respect to each other, and a connector portion 50. FIG. 2A is a perspective view of the body of the device. FIG. 2B is a side view of the body of the device showing slots 46 to be cut from which struts will emerge. FIG. 2C is a cross-sectional view of the device. FIG. 2D is a perspective view of the device after the struts 40 have radially expanded. FIG. 2E is a side view of the device after the struts have radially expanded. FIG. 2F is a cross-sectional view through the struts of the device after the struts have radially expanded.

FIGS. 3A-3F provide views of a fracture-stabilization device 20 with a rectangular body having a lumen 54 and two anchorable members 30, each with four radially expandable struts 40′, each emanating from a slot 46 cut through a flat surface of the body and expanding with a leading sharp edge 42, and a connector portion 50. FIG. 3A is a perspective view of the body of the device. FIG. 3B is a side view of the body of the device showing slots 46 to be cut from which struts will emerge. FIG. 3C is a cross-sectional view of the device. FIG. 3D is a perspective view of the device after the struts 40 have radially expanded. FIG. 3E is a side view of the device after the struts have radially expanded. FIG. 3F is a cross-sectional view through the struts of the device after the struts have radially expanded.

FIGS. 4A-4F provide views of a fracture-stabilization device 20 with a rectangular body having a lumen 54 and two anchorable members 30, each with two radially expandable struts 40′ emanating from length-wise cuts in a flat surface of the body and expanding with a leading flat edge, and a connector portion 50. FIG. 4A is a perspective view of the body of the device. FIG. 4B is a side view of the body of the device showing slots 46 to be cut from which struts will emerge. FIG. 4C is a cross-sectional view of the device. FIG. 4D is a perspective view of the device after the struts 40 have radially expanded. FIG. 4E is a side view of the device after the struts have radially expanded. FIG. 4F is a cross-sectional view through the struts of the device after the struts have radially expanded.

FIGS. 5A-5F provide views of a fracture-stabilization device 20 with a rectangular body having a lumen 54 and two anchorable members 30, each with two radially expandable struts 40′ emanating from length-wise cuts at a vertex of the rectangle, each strut expanding with a leading sharp edge 42, and a connector portion 50. FIG. 5A is a perspective view of the body of the device. FIG. 5B is a side view of the body of the device showing slots 46 to be cut from which struts will emerge. FIG. 5C is a cross-sectional view of the device. FIG. 5D is a perspective view of the device after the struts have radially expanded. FIG. 5E is a side view of the device after the struts 40 have radially expanded. FIG. 5F is cross-sectional view of through the struts of the device after the struts have radially expanded. Device embodiments such as these depicted in FIG. 5, FIG. 4, and FIG. 9 with two radially expandable struts may be particularly advantageous for fixing fractures in a flat bone such as a skull plate (FIG. 14) or in any bone or fracture site that is small, or has a narrow planar constraint.

As mentioned above, although the examples shown in FIGS. 1A and 2A are fracture fixation devices that are integrally formed, the anchorable regions may be separate and attachable including separate and attachable to a connector) via the connector region. Further, any of embodiments described herein may include one or more attachment regions for attachment to a delivery device (including both distal and proximal attachment sites), and attachment to a length-adjusting device (for changing the spacing between the anchorable members), or attachment to a source of flowable material (e.g., cement). Attachment sites may be threaded attachment sites, interlocking attachment sites (e.g., keyed attachment sites), gripping attachment sites, or any appropriate releasable attachment site.

FIGS. 6-8 show exemplary anchorable members 30 which may be understood as components of a complete double-anchored device 20, these single anchorable members being presented to exemplify particular features comparative way. FIG. 6 provides a view of a single anchorable member 30 with two radially opposed struts 40 in an expanded configuration, the member being a component joinable with a connector portion and a second anchor to form a complete fracture fixation device. FIG. 7 provides a view of a single anchorable member 30 with three radially distributed struts 40 in an expanded configuration, the member being a component joinable with a connector portion and a second anchor to form a complete fracture fixation device.

FIG. 8 provides a view of a single anchorable member 30 with four radially opposed struts 40 in an expanded configuration, the member being a component joinable with a connector portion and a second anchor to form a complete fracture fixation device, the anchorable member further including a central rod or tube 54 that forms a continuous passageway with a connector in the fully assembled device. In some variations, the connector is the central tube 54 shown, and the anchorable members 30 may be slidable thereon. The anchorable members may be locked into position. In some variations, the connector does not lock to the anchorable members. The connector portion and/or the rod may include holes 52 from which a flowable bone cement may be ejected. Lumen 54 as seen in FIG. 8 in the form of a central rod extending through the anchorable member 30 may also be understood as to include the contiguous open space, in general, within the interior of expanded struts 40 as depicted in FIG. 6 and FIG. 7.

FIGS. 9A and 9B provide views of a fracture-stabilization device 20 with a rectangular body and two anchorable members 30, each with two radially expandable struts emanating from length-wise cuts at a vertex of the rectangle. This device is similar to that depicted in FIG. 5 except that the corners of the rectangle have been pinched or crimped in, giving the corner an internal angle more acute than 90 degrees. These acute corners become the leading and cutting edge 42 of a strut 40 as it expands, and in this embodiment the leading edge is particularly sharp. FIG. 9A is a perspective view of the body of the device. FIG. 9B is a view of one strut of the device after radial expansion.

FIGS. 10A-10F show a portion of one anchorable member of an embodiment of a double-anchored fracture fixation device with a linearly corrugated or crenellated surface, from which nine expandable struts 40′ emanate. FIG. 10A shows the anchorable member 30′ in a linearly constrained, non-radially expanded configuration. Slots 46 are present in the inner vertex of corrugations. FIG. 10B shows the anchorable member 30″ with expansion of the struts 40″ to a first position, which may either be a partial or fully self-expanded configuration, depending on the preferred configuration of the heat-treated shape memory metal. FIG. 10C shows expansion the anchorable member 30 and the expandable struts 40 to a second position, more expanded than the first position of FIG. 10B. FIG. 10D shows a radial cross sectional view of anchorable member 30′ at position 10D of FIG. 10A, showing the corrugated nature of the body of the anchorable member. FIG. 10E shows a radial cross sectional view of anchorable member 30″ at position 10E of FIG. 10B, showing the M-shaped cross-sectional profile the expanded or partially-expanded struts 40″. FIG. 10F shows a radial cross sectional view of anchorable member 30 at position 10F of FIG. 10C, showing the flattened M-shaped cross-sectional profile of fully expanded struts 40.

FIGS. 11A-11D show one example of a fracture-stabilization device that has been exploded into three parts, as well as cross sectional views of the body of the device, and of the anchorable members in their expanded configuration. This figure may illustrate the location of various dimensions of the device. Dimensions of anchorable members 30 of a fracture stabilizing device 20 may be chosen according to their intended site of use. The exemplary dimensions provided here are to help in providing an understanding of the invention, and are not intended to be limiting. For example, in some embodiments, the length L of the body 45 of an anchorable member when the struts 30 are in the radially expanded configuration may vary from about 7.5 mm to about 48 mm, and in particular embodiments, from about 24 mm to about 40 mm. In other embodiments, for particular applications, the length of the body may be less than 7.5 mm or greater than 48 mm. The thickness T (FIG. 11B) of the tube wall of a tubular body 45 may vary from about 0.2 mm to about 2.5 mm, and in typical embodiments is about 0.5 mm in thickness. The outside diameter D1 of the body of the device in its linear configuration may vary. In one variation, the outer diameter varies between about 1 mm to about 8 mm in diameter. FIG. 11D shows a cross sectional view of an alternative embodiment with three struts, radially distributed at 120 degrees, is included to convey the applicability of this diameter measurement even when struts do not form a straight-line diametric structure as can four struts. In the context of a released or anchoring configuration of an anchorable device 30, the struts 40 may expand to a maximal radial distance (FIGS. 11C and 11D) from about 3.5 mm to about 22 mm, to create a maximal diameter D2 (extrapolating the strut profiles to form a circle enclosing the maximal points of expansion) of about 7.5 mm to about 44 mm. In other embodiments, for particular application to particular fracture sites, the maximal expansion diameter may be less than 4 mm or greater than 25 mm.

FIGS. 12A-12E show views of the deployment of a fracture-stabilization device into a hip, passing through a point just below the greater trochanter of the femur, through a proximal region of bone 131, across the fracture 130, and into a distal region of bone 132 within the head of the femur. FIG. 12 also shows the distribution of cancellous bone 101 and cortical or dense bone 102 within the femur. FIG. 12A shows a drill bit 103 forming a passageway for the device; such drilling typically occurs after aligning the fracture so as to restore the fractured bone to its natural position, or to a position that best approximates the natural position. Such alignment may be attained by methods well known in the art, including the use of a goniometer. FIG. 12B shows the formed passageway 105 prepared to receive the device. FIG. 12C shows a delivery device 71, in this example, a cannula or a device with a distal portion that includes a sleeve that radially envelopes the device, being inserted into the passageway, and positioning the distal anchorable member of a device. FIG. 12D shows the first or distal anchorable member after expansion of the struts after the first or distal anchorable member 30a of the device has been partially pushed out of the distal end of the cannula 71, while at the same time, the cannula has been partially withdrawn from the implant site. The exemplary device in this series of figures is being inserted in its complete form, i.e., with the first anchorable member 30a, the connector 50, and the second anchorable member 30b already either conjoined prior to implantation, or the device as a whole fabricated as single integrated device. In some variations of the fracture-fixation devices that are delivered by a cannula, the anchorable members of the device may be self-expand upon release from the radial constraints of the cannula. Further, in such method embodiments, the first anchorable member expands first, and the second expandable member expands second. FIG. 12E shows the device in situ, the proximal or second expansion member after its struts have expanded, and after the two anchorable members of the device have been drawn together, tightening the fracture zone 130. Details of drawing two anchorable members together after implantation of a device are shown in FIG. 16, as described below.

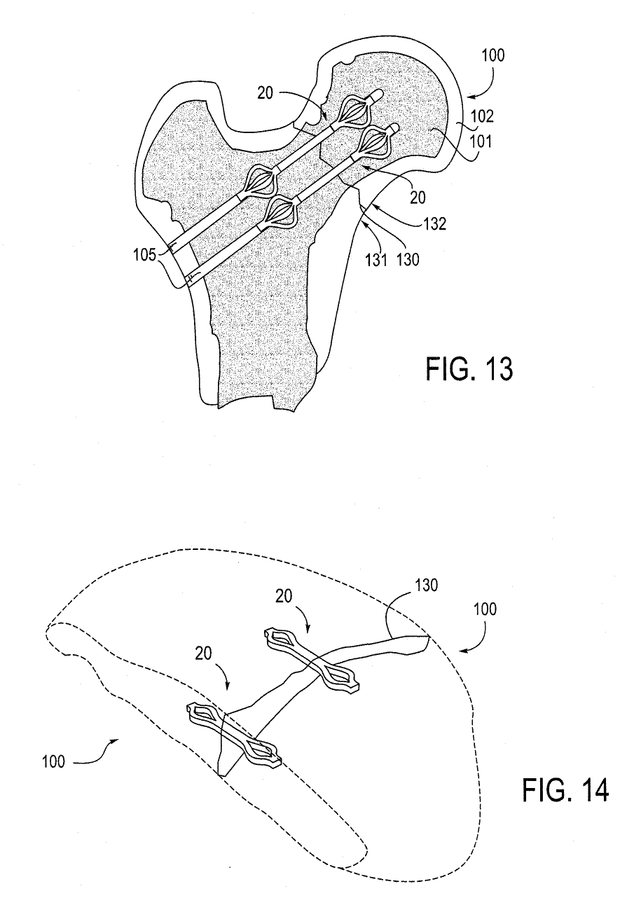

Some fractures may benefit from the implanting of more than one fracture-stabilization device. In these instances, each device needs to have preparatory drilling to form a passageway and implanting in a manner similar to that detailed for a single device as in FIG. 12. FIG. 13 depicts two fracture-stabilization devices implanted into a fracture of a hip in location similar to that depicted in FIG. 12.

FIG. 14 depicts two fracture-stabilization devices implanted into a flat bone 100 such as a skull plate; the devices 20 are substantially flat in their expanded profile as the expandable members each have two coplanar struts (such as those depicted in FIG. 4, 5, or 9).

FIG. 15 depicts an embodiment of a fracture-stabilization system that is in the form of a kit 10, the kit including an Allen head tool 53 shown in a side view and a perspective view, a first anchorable member 30a and a second anchorable member 30b, a connector 50, a delivery device 70, a container of a flowable bone filling composition 61, and an applicator, including a first rod 55 for engaging the first or distal anchor 30a, and a second (outer) rod 56 for engaging the second or proximal anchor 30b. The two rods of the delivery system may constrain the anchorable members from expanding during deployment. After delivery, one or both rods may be withdrawn, allowing anchorable members to contract and radially self-expand into anchoring configurations.

The delivery device 70 in this example has a distal threaded portion 72 that engages threads 58a on the first anchorable member 30a. The first anchorable member 30a has a connecting region (rod engaging feature 53a) that engages plug 59 on rod 55. The second anchorable member has a connecting region (rod engaging 53b) that engages plug 59 on rod 56. Rod 56 further has a stop bar 62 that meets the interior of the distal end of the second anchorable member and a plug mount 63 with plugs 59 that engage the proximal end of the second anchorable member. Rods 55 and 56 may both be considered embodiments of a length-constraining rod, which may constrain the length (in this case, prevent contraction) of an anchorable member, by engaging in a releasable way either or both the proximal or distal portion of an anchorable member in such a way that contraction of the member is prevented. The releasable-engagement means that interact between an anchorable member and a length-constraining rod may be of any suitable type. In the particular embodiments shown, the feature on the rods are male plugs that can rotate into female slots within the anchorable members, but the male-female orientation may be reversed in some embodiments, or more generally be of any suitable mechanism. Connector 50 has threaded portion 57a that engages threads 58a on first anchorable member 30a, and connector 50 also has threads 57b that engage threads 58b on second anchorable member 30b. Connector 50 further has an Allen head female feature 51 that engages the male head on Allen tool 53. The threads 57a and 57b of the connector and their respectively engaging threads on the respective anchorable members are configured oppositely such that the connector 50 acts like a turnbuckle when turned by the Allen tool 53, and can thus pull the anchorable members together or extend them further apart.

FIGS. 16A-16O illustrate of a fracture fixation device in three conjoinable units (first and second anchorable members and a connector portion), and describe assembly and insertion of one variation of the fracture-fixation device. In this sequence of figures, the device is shown variously both in isolation, ex situ, and as implanted across a fractured region within the neck of a femur. FIG. 16A shows the first anchorable member 30a′ in a constrained configuration, held by the delivery device including opposition rod 55. The anchorable member is shown threadably-connected to the outer rod of the delivery device 70 (also a hollow element), and the connector element is also secured to the delivery device, so that the distal anchorable member is held in the collapsed configuration as tension is applied.

FIG. 16B shows the first anchorable member after opposition rod 55 has been partially withdrawn, releasing the applied tension and allowing the anchorable member to at least partially self-expand.

FIG. 16C shows the first anchorable member being further expanded by a mechanical assist. The opposition rod 55 has been re-engaged (or has remained engaged) at the distal portion 59 of the first expandable member 30a, and the distal portion is being pulled proximally by rod 55. This is an optional step in the implantation of the device, and an analogous step may be taken with regard to the second or proximal anchorable member. Although the anchorable members are self-expanding, and expand to a preferred configuration when their expansion is unimpeded, when implanted in bone, such expansion can meet variable amounts of resistance. For this reason, under some conditions, it may be desirable to mechanically assist in expansion of the struts of the anchoring configuration of an anchorable member. An analogous mechanical expansion step and a tool for such has been described in U.S. patent application Ser. No. 11/468,759.

FIG. 16D continues as if the step shown in FIG. 16C had not been taken, and shows the first anchorable member released from the linearly constraining opposition rod 55 and in a self-expanded configuration, rod 55 now withdrawn from the first anchorable member 30a. The second (proximal) anchorable member may then be applied by detaching the threaded delivery device, and inserting a connector, as shown in FIG. 15 50. The connector can be threaded or otherwise attached to the end of the first anchorable member. A second anchorable member can them be inserted by holding it in tension and attaching it (e.g., via threads) to the proximal end of the connector. Once it has been connected, the tension may be released, and it may be allowed to self-expand. The connector can be adjusted to change the spacing between the expanded anchorable members, as described above. During this process the fracture-fixation device has maintained a central passageway.

FIG. 16E shows a similar first anchorable member 30a in situ in the self-expanded configuration, as it is anchored in its expanded configuration. It can be seen that the anchorable member positioned in a region of bone 132 that is proximal to fracture 130, with respect to the path of entry 105. The anchorable member is embedded in cancellous bone region 101 of a femoral head 100, which is encased in cortical bone 102.

FIG. 16F is an in situ view showing the connector portion 50 being threadably connected to the first anchorable member 30a, the connector being deployed by a delivery device, the delivery device threadably connecting the connector 50 to the first anchorable member 30a.

FIG. 16G shows first anchorable member 30a and the connector 50 now conjoined, and a second anchorable member 30b′ now being brought into position to engage the connector portion 50 by a delivery device with a rod 55 constraining the anchorable member 30b′ in its linear configuration.

FIG. 16H shows the second anchorable member 30b′ now in contact with the connector 50, the member still being linearly constrained by the rod 56 of the delivery device. More specifically, it can be seen that stop bar 62 and the plugs 59 on mount 63 of rod 56 are preventing the linear contraction (and consequent radial expansion) of anchorable member 30b′.

FIG. 16I shows the second anchorable member 30b now released from its proximal attachment to the rod (the rod 56 has been rotated, releasing plugs 59 from their engagement site at the proximal end of anchorable member 30b), and the anchorable member 30b has now linearly contracted and radially expanded.

The exemplary deployment sequence just described is one in which the first anchorable member expands first, after implantation as a single piece, and then a connector is added, and then the second anchorable member, which then radially expands. Other embodiments of the inventive method include implantation of a device that is assembled in situ, but delaying the expansion of the first anchorable member until assembly of the device is complete, and then expanding the two anchorable members simultaneously, or nearly simultaneously. In other embodiments of system and method as described above and shown in FIGS. 12A-12E), a fully assembled or integrally formed device is implanted and the anchorable member then each radially-expanded synchronously.

FIG. 16J is an in situ view showing the second anchorable member 30b now implanted and expanded, and the deploying rod 56 now withdrawn from the implant site. Both anchorable members are now expanded in their anchoring configuration, however the fracture gap 131 has not yet been tightened by the drawing closer of the two anchorable members 30a and 30b.

FIG. 16K shows an Allen wrench connector deployer 53 extending through the second anchorable member 30b to engage the connector at Allen female feature 51 within connector 50 and beginning to rotate the connector with respect to the two anchorable members, drawing them closer together, as indicated by the directional arrows.

FIG. 16L shows the first 30a and second 30b anchorable members now drawn together by a turn-buckle rotation of the connector 50 threadably engaging both the first and second anchorable members in a turnbuckle manner. The pulling together or approximating of the anchorable members may be complemented by the reverse action, a distraction or separating of the anchorable members, as may be required or desired in some procedures. Further, such manipulations may be done before expansion of one or both of the anchorable members.

FIG. 16M is an in situ view showing the fully assembled device 20 in its anchoring configuration, the two anchorable members drawn together to the desired degree toward the connector, the fracture regions 131 and 132 also drawn together, and the deployment device having been withdrawn.

FIG. 16N shows an injector tube 62 connected to the proximal end device 20, engaging a connection on anchorable member 30b, and injecting a flowable cementing composition 61 through the passageway 54 extending through the device. The cementing composition 61 is being emitted into the space within the expanded struts of the anchoring members 30a and 30b, and through holes 52 in connector 50 to emerge into available space within bone that is peripheral to the connector.

FIG. 16O shows the device, the cement inserter removed, the device now fully implanted, and stabilized by the cement 61 now hardened.

FIGS. 17A-17E show various embodiments of fracture fixation devices that have dissimilar first and second anchors for custom fitting into fracture sites. FIG. 17A shows a device with a three-strut anchorable member and a two-strut anchorable member, in each case that struts curvilinear and asymmetrically bowed. FIG. 17B shows a device with a two-strut anchorable member and a four-strut anchorable member, in case the struts are symmetrically bowed and having substantially straight segments. FIG. 17C shows a device with a four-strut anchorable member that is significantly larger than its two-strut companion. FIG. 17D shows a device with three anchorable members, each member having two struts, the members expanding in different radial orientations, two with substantially straight segments in the struts, and a third with curvilinear struts. FIG. 17E shows a device with an anchorable member having two asymmetrically bowed struts and a central hollow rod and a second anchorable member with four symmetrically bowed struts and without a central rod.

Although the fracture fixation devices described herein typically include two anchorable (expandable) regions separated by a connector region, other variations are encompassed by this disclosure, including devices having more than two anchorable regions. For example, a series of interconnected expandable regions could form a fracture-fixation device. In addition, the connector regions could be formed of bendable, or rotatable material. In some variation the connector region or component is adjustable to shorten or lengthen the spacing between them without rotating them. For example, the connector region may be an interlocking telescoping region.

While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A system for stabilizing a bone fracture, comprising:
a first anchorable member and a second anchorable member, each member having a central passageway, each member having a constrained non-anchoring configuration and a released anchoring configuration; and
a connector having a central passageway, the connector configured to attach to the proximal end of the first anchorable member and the distal end of the second anchorable member, such that the central passageways of the anchorable members and the connector form a continuous passageway.

2. The system of claim 1, wherein the released anchoring configuration includes a radially expanded structure.

3. The system of claim 1, wherein the first anchorable member and the second anchorable member and the connector are separable.

4. The system of claim 1, wherein the connector and one of the first anchorable member or the second anchorable member are conjoined.

5. The system of claim 1, wherein the first anchorable member, the connector, and the second anchorable element are conjoined.

6. The system of claim 1, wherein the non-anchoring configuration of the anchorable members includes three or more flat surfaces in cross section.

7. The system of claim 1, wherein the non-anchoring configuration of the anchorable members is rectangular in cross section.

8. The system of claim 1, wherein the released anchoring configuration of the anchorable members includes at least one cutting surface.

9. The system of claim 1, wherein the released anchoring configuration of the anchorable members includes radially expanded struts

10. The system of claim 9, wherein the radially-expanded struts form a symmetrical bow.

11. The system of claim 9, wherein the radially-expanded struts form an asymmetrical bow.

12. The system of claim 11, wherein the asymmetrical bow has its greatest radial diameter distributed proximally.

13. The system of claim 1, wherein the passageways of the first anchorable member, the connector, and the second anchorable member are adapted to convey a flowable material.

14. The system of claim 1, wherein the connector includes holes adapted to allow egress of a flowable material.

15. The system of claim 1, wherein the first anchorable member includes an attachment site at its distal end configured to releasably attach to a delivery device.

16. The system of claim 1, wherein the connector and at least one of the first or second anchorable members is threadably connected such that rotation of at least one of the anchorable members with respect to the connector changes the distance between the two anchorable members.

17. The system of claim 1, further comprising a delivery device for positioning the first anchorable member, the connector, and the second anchorable member into a bone fracture site.

18. The system of claim 17, wherein the delivery device is configured to be releasably attached to the distal end of first anchorable member.

19. The system of claim 17, further comprising a rod configured to extend distally from the delivery device into the continuous passageway, the rod further configured to attach to the distal end of the first anchorable member.

20. The system of claim 17, wherein the delivery device comprises a sleeve that radially encloses the first anchorable member, the connector, and the second anchorable member.

21. The system of claim 20, further comprising a push rod configured to extend distally from the applicator to the proximal end of the second anchorable member.

22. A method for stabilizing a fractured bone, comprising:
forming a passage in the fractured bone through a proximal bone region, across the fracture, and into a distal bone region;
positioning a bone fracture-stabilizing system in the passage, the system including:
a first anchorable member and a second anchorable member, each member having a central passageway, each member having a constrained non-anchoring configuration and a released anchoring configuration; and
a connector having a central passageway, the connector configured to attach to the proximal end of the first anchorable member and the distal end of the second anchorable member, such that the central passageways of the anchorable members and the connector form a continuous passageway; and
anchoring the first anchoring member within the distal bone region and the second anchoring member within the proximal bone region.

23. The method of claim 22, further comprising aligning the proximal bone region and the distal bone region prior to forming the passage in the bone.

24. The method of claim 22, further comprising inserting the anchorable members into the passage in the constrained configuration.

25. The method of claim 22, further comprising releasing the anchoring members from a constrained configuration by disengaging the first anchoring member from a rod.

26. The method of claim 22, further comprising radially expanding a plurality of bowed struts from each anchorable member to anchor the member within the bone.

27. The method of claim 26, wherein the struts radially self-expand.

28. The method of claim 26 wherein the struts are expanded with a mechanical assist after self-expanding.

29. The method of claim 22, further comprising simultaneously expanding the first and second anchorable members.

30. The method of claim 22, further comprising expanding the first anchorable member before expanding the second anchorable member.

31. The method of claim 22, further comprising exposing cutting surfaces on bowed struts forming the first and second anchorable members.

32. The method of claim 22, further comprising flowing a bone-filling material through the continuous passageway.

33. The method of claim 32, further comprising hardening the bone-filling material as to form a solid material.

34. The method of claim 22, further comprising flowing a bone-filling material through the continuous passageway so that at least some material exits holes from the connector.

35. The method of claim 22, further comprising drawing the anchorable members closer together by rotating the connector.
